(19) United States
(12) Patent Application Publication
(10) Pub. No.: US 2015/0343233 A1
(43) Pub. Date: Dec. 3, 2015

(54) METHOD AND APPARATUS FOR TREATING CARDIAC ARRHYTHMIAS USING ELECTROMAGNETIC RESYNCHRONIZATION

(71) Applicants: Eli NHAISSI, OLD WESTBURY, NY (US); ASHER HOLZER, RA'ANANA (IL); JACOB DAGAN, TEL AVIV (IL); AEROTEL LTD., HOLON (IL)

(72) Inventors: MICKEY SCHEINOWITZ, KFAR SABA (IL); ELI NHAISSI, OLD WESTBURY, NY (US); ASHER HOLZER, RA'ANANA (IL); JACOB DAGAN, TEL AVIV (IL)

(73) Assignee: AEROTEL LTD., HOLON (IL)

(21) Appl. No.: 14/768,599

(22) PCT Filed: Mar. 28, 2014
(86) PCT No.: PCT/US14/32104

§ 371 Date: Aug. 18, 2015

Related U.S. Application Data
(60) Provisional application No. 61/813,733, filed on Apr. 19, 2013.

(51) Int. Cl. A61N 2/00; A61N 1/39; A61N 2/02

(52) U.S. Cl. CPC A61N 2/002; A61N 2/004; A61N 2/02; A61N 1/3987

(57) ABSTRACT

A method and an apparatus for the treatment of cardiac arrhythmias using a weak pulsed magnetic field. A transducer that emits electromagnetic radiation of a prescribed frequency and peak intensity is placed on the patient's chest and, as a result, the weak electromagnetic field can cause activation, reactivation, inhibition or remodeling of electrophysiological change in cardiac tissue in an irradiated heart. This treatment method has wide application for use in patients who experience cardiac arrhythmia.

### BACKGROUND

The present disclosure relates generally relates to the treatment of patients having treatable medical conditions using weak (i.e., low-intensity) low-frequency electromagnetic fields.

The term “arrhythmia” (i.e., irregular heartbeat) encompasses any of a large and heterogeneous group of conditions in which there is abnormal electrical activity in the heart. A heartbeat that is too fast is called tachycardia and a heart beat that is too slow is called bradycardia. Although many arrhythmias are not life-threatening, some can cause cardiac arrest.

Atrial fibrillation is the most common cardiac arrhythmia. In atrial fibrillation, the normal regular electrical impulses generated by the sinoatrial (SA) node are overwhelmed by disorganized electrical impulses usually originating in the roots of the pulmonary veins, leading to irregular conduction of impulses to the ventricles which generate the heartbeat. Atrial fibrillation may be treated with medications to either slow the heart rate to a normal range (“rate control”) or revert the heart rhythm back to normal (“rhythm control”). Synchronized electrical cardioversion can be used to convert atrial fibrillation to a normal heart rhythm. Synchronized electrical cardioversion uses metallic plates with conductive gel to deliver a therapeutic dose of electric current to the heart at a specific moment in the cardiac cycle. A synchronizing function (either manually operated or automatic) allows the cardioverter to deliver a reversion shock of a selected amount of electric current over a predefined number of milliseconds at the optimal moment in the cardiac cycle which corresponds to the R wave of the QRS complex on the electrocardiogram (ECG). Synchronized electrical cardioversion is used to treat atrial fibrillation, atrial flutter, and ventricular tachycardia, when a pulse is present.

In the past, the possibility of treating cardiac arrhythmias with the application of low-intensity, low-frequency electromagnetic fields has been proposed. In particular, the concept of using an electromagnetic field generator as a regulator of atrial fibrillation has been previously disclosed.

The heart is a precise oscillatory organ capable of generating uninterrupted rhythmical activity over a very long period. The SA node is the impulse-generating (pacemaker) tissue located in the right atrium of the heart, and thus is the generator of normal sinus rhythm. The pacemaker cells located in the SA node are specialized cardiac myocytes that generate the regular oscillatory action potentials that drive each contraction cycle. In muscle cells, an action potential is the first step in the chain of events leading to contraction. Action potentials are generated by special types of voltage-gated ion channels embedded in a cell's plasma membrane. These channels are shut when the membrane potential is near the resting potential of the cell, but they rapidly begin to open if the membrane potential increases to a threshold value. When the channels open, they allow an inward flow of sodium ions. The rapid influx of sodium ions causes the polarity of the plasma membrane to reverse, and the sodium ion channels then rapidly inactivate. Potassium channels are then activated, and there is an outward current of potassium ions, returning the electrochemical gradient to the resting state. The pacemaker function depends upon the interaction between the foregoing plasma membrane ion channels. For example, malfunction of potassium channels may cause life-threatening arrhythmias.

Atrial fibrillation is the single most important cause of ischaemic stroke in people more than 75 years of age. Atrial fibrillation (AF) is characterized by rapid and irregular activation of the atrium, for example, 400-500 pulses of the atrium muscular wall per minute in humans. The occurrence of AF increases with age, with a prevalence rising from 0.5% of people in their 50s to nearly 10% of the octogenarian population. Several cardiac disorders predispose to AF, including coronary artery disease, pericarditis, mitral valve disease, congenital heart disease, congestive heart failure (CHF), thyrotoxic heart disease and hypertension

Normally, the heart rate is finely attuned to the body's metabolic needs through physiological control of the cardiac pacemaker function of the SA node, which maintains a rate of about 60-90 beats per minute at rest and can fire as rapidly as 170-200 times per minute at peak exercise. During AF, atrial cells fire at rates of 400-500 times per minute.

It is now accepted that the effect of the magnetic field on an excitable cell's membrane works through influencing the kinetics of calcium ions. This happens in the neurons as well as in the cardiac myocytes (cardiac muscle cells), which generate the electrical impulses that control the heart rate. Field intensity and modulation frequency were shown to be important determinants in weak magnetic fields causing cellular Ca²⁺ efflux. The Ca²⁺ channel modifies other ion transporters, such as the potassium and sodium channels.

Studies on animal neurons showed that 86% of the magnetically sensitive cells were inhibited by a weak magnetic field and 14% were excited. Both effects resulted from the movements of Ca²⁺ ions at the cell membrane (Azanza and del Moral, 1988). It is known that outward immigration of K⁺ ions through channels opened by Ca²⁺ fluctuations brings forth hyperpolarization of the cells wherever they exist. This is followed by efflux of the K⁺ ions triggered by the inside shift of Ca²⁺, which may activate the cell action potential (Meech, 1978).

Thus magnetic fields induce movements of Ca²⁺ ions across the cell membrane, which affects the shifts of K⁺ ions through openings in their membrane channels. The cell may become either inhibited or excited, depending on its inherent properties and most probably also depending on the specific pattern of weak magnetic field (WMF) stimulation.

Among the diverse excitable cells within the heart are the highly specialized pacemaker cells (in the SA node and the AV node, which have spontaneous depolarization due to slow outward efflux of K⁺ ions, until reaching the threshold of excitation). Atrial cells, and ventricular cells, all have different electrophysiological properties, yet all possess Ca²⁺ channels (in addition to Na⁺ and K⁺ channels). But, in a pathological state, all may exhibit an automatic excitability of their own to fire rapidly or irregularly, causing cardiac arrhythmias. This is one mechanism of cardiac arrhythmia.

A weak electromagnetic field (as weak as is still capable of affecting the flux of Ca²⁺ ions across the cell membranes) can ignite a self-propagated process of Ca²⁺, K⁺ and Na⁺ ion shifts. It depends on the modes of WMF stimulation (frequency, intensity and configuration) and/or an additional external intervention (such as the application of drugs), to determine if the cell will discharge following its excitation or will be further inhibited. It is known from in vitro experiments that weak magnetic fields (VWMF) can induce activation, reactivation and inhibition of the excitable cells. Weak magnetic fields can have a negative cronotropic effect on cardiac pacemaker cells and can be used continuously or intermittently to alleviate atrial fibrillation. The effect of WMF to promote calcium efflux from atrial and myocardial cells is of utmost importance in arresting the deterioration observed with patients suffering from atrial fibrillation.

Accordingly, there is a need for systems and methods for treating cardiac arrhythmias using weak electromagnetic fields.

### SUMMARY

The subject matter disclosed herein is directed to a method and an apparatus for monitoring a patient's cardiovascular system and, upon detecting an arrhythmia, applying weak (i.e., 10 picotesla to 25 nanotesla) electromagnetic fields that induce effects to ameliorate the defective cardiac performance. The apparatus comprises an electromagnetic resynchronization (EMR) device, which may optionally be coupled to a defibrillator, which can be activated by the EMR device in the event that ventricular fibrillation is detected. As described below, the EMR device comprises an ECG monitoring system and an electromagnetic field generator.

In accordance with embodiments disclosed herein, the ECG monitor/analyzer is programmed to issue an activation signal in response to detection of a cardiac arrhythmia (e.g., atrial fibrillation and ventricular tachycardia); and the electromagnetic field generator is programmed to apply a pulsed low-intensity, low-frequency magnetic field to a patient's heart (including, in particular, the SA node) in response to the activation signal from the ECG monitor/analyzer. The activation signal is encoded to include information or a characteristic that indicates which cardiac arrhythmia has been detected by the ECG monitor/analyzer. It should be appreciated that the system and methodology disclosed herein may be adapted to treat cardiac arrhythmias other than atrial fibrillation and ventricular tachycardia.

The electromagnetic field generator can be programmed to generate a first pulsed low-intensity magnetic field having a peak intensity in a first peak intensity range and a frequency in a first frequency range in response to a first activation signal from the ECG monitor/analyzer indicating that an atrial fibrillation event is occurring. In addition, the electromagnetic field generator can be programmed to generate a second pulsed low-intensity magnetic field having a peak intensity in a second peak intensity range and a frequency in a second frequency range in response to a second activation signal from the ECG monitor/analyzer indicating that a ventricular tachycardia event is occurring. This principle of operation can be extrapolated to encompass the design of further alternative magnetic field generation protocols for other types or sub-types of arrhythmia. A set of protocols may have different (yet overlapping) pulsed magnetic field peak intensity ranges and different (yet overlapping) pulsed magnetic field frequency ranges.

In accordance with embodiments disclosed herein, the electromagnetic field generator comprises coils which are placed in proximity to the patient's heart. The magnetic field generator is programmed to produce a pulsed electromagnetic field, preferably synchronized with the cardiac cycle. The pulsed electromagnetic field will have a peak intensity and a frequency appropriate for resynchronizing pathological/non-synchronized cells (e.g., cardiac cells, cardiac myocytes), which peak intensity and frequency (as previously stated) may fall in respective ranges which are dependent on which cardiac arrhythmia has been detected.

In accordance with further embodiments, the magnetic wave generator can be programmed to produce, in succession, pulsed electromagnetic fields having different frequencies. The ECG monitor/analyzer would monitor the resulting ECG data and analyze which frequency produced the optimum response from the patient. The ability to scan the frequencies in the frequency range applicable to the cardiac arrhythmia which has been detected allows the system to determine an optimum frequency. The electromagnetic wave generator would then supply pulsed electric current to the coil array at this optimum frequency, maintaining this frequency for a longer period of time.

In addition, research has suggested that an alternating magnetic field may influence the mechanical vibration of the myocardium cell membranes, and thus might influence the conductivity of ions through the membrane. Influence means not only intensity (the number of ions going through) but the duration the channel is open. Thus as a non-limiting example, the EMR device may be triggered and gated to deliver energy during part of the ECG cycle; for example, only during the QRS period (about 80 millisecond, which would be only about two cycles of EMR pulsed current depending on the frequency.

The EMR device disclosed herein has the capability to resynchronize the cardiac cells by shortening the action potential duration via opening of potassium ion channels on the cell membranes. This EMR device has no effect on normal, synchronized cells. The EMR device works mostly on pathological/non-synchronized cells to gradually resynchronize all non-synchronized cells. Thus, compared with a defibrillator that provides a high-voltage, abrupt energy to cease the arrhythmia, the EMR device accomplishes the same, but with no effect on normal cells, thereby minimizing the overall damage to the human body. It is a resynchronization device intended for non-life-threatening cardiac arrhythmias. Therefore, it is not employed in cases of ventricular fibrillation. The EMR device can be a stand-alone device operated by the patient. It can be operated by a physician, in ambulances, physician's clinics, hospitals. It can also be implanted into the patient in a miniaturized configuration.

A mode of operation for the overall system may be as follows: Electrocardiographic monitoring is utilized to detect cardiac arrhythmia in a patient. Once cardiac arrhythmia has been detected, an ECG monitor/analyzer will signal/switch-on the electromagnetic field generator for several minutes (e.g., 10 to 60 minutes). The electromagnetic field generator will deliver a low-frequency (i.e., 2 to 60 Hertz) electromagnetic field having a peak magnetic field strength (in the volume of space occupied by the SA node of the patient's heart) in the range of 10 nanotesla to 900 picoteslas. The ECG monitor/analyzer will detect once the arrhythmia has ceased and signal the electromagnetic field generator to stop. If the arrhythmia has not ceased, the ECG monitor/analyzer will signal the electromagnetic field generator to change frequency (±5 Hz) for another session. If during the second therapeutic session, the arrhythmia still does not cease, the ECG monitor/analyzer will signal the patient to see his doctor or change the therapeutic routine.

The above-described EMR device can detect the arrhythmia as soon as it starts and then apply an electromagnetic field to the patient's chest without delay, not shocking him as a regular defibrillator does, but rather, by applying this unnoticed electromagnetic field, suppress the arrhythmia as soon as it is detected. The EMR device is comfortable, easy to operate, customizable, and financially affordable and can be used as a long-term (months) Holter monitor to follow up the patient. The system optionally (but preferably) includes a regular defibrillator in case the patient's heart experiences ventricular fibrillation (which is a life-threatening event). In that event, the ECG monitor/analyzer will send an activation signal to the defibrillator, which will initiate the delivery of an electric shock. It will not be necessary in most of the applications to attach the defibrillator pads to the patient, only in cases when the physician anticipates such an event occurring with a specific patient.

In addition, the system includes an ECG and event recorder, which will store all of the patient's ECGs and the initiation and cessation times for the application of the EMR or the defibrillator.

Other aspects and further details of the systems and methods for treating cardiac arrhythmias generally described above are disclosed below.

### BRIEF DESCRIPTION OF THE DRAWINGS

FIG. 1 is a diagram showing the configuration of an apparatus treating cardiac arrhythmia in accordance with one embodiment.

FIG. 2 is a block diagram representing circuitry incorporated in a non-invasive electromagnetic resynchronization device in accordance with another embodiment.

Reference will hereinafter be made to the drawings in which similar elements in different drawings bear the same reference numerals.

### DETAILED DESCRIPTION

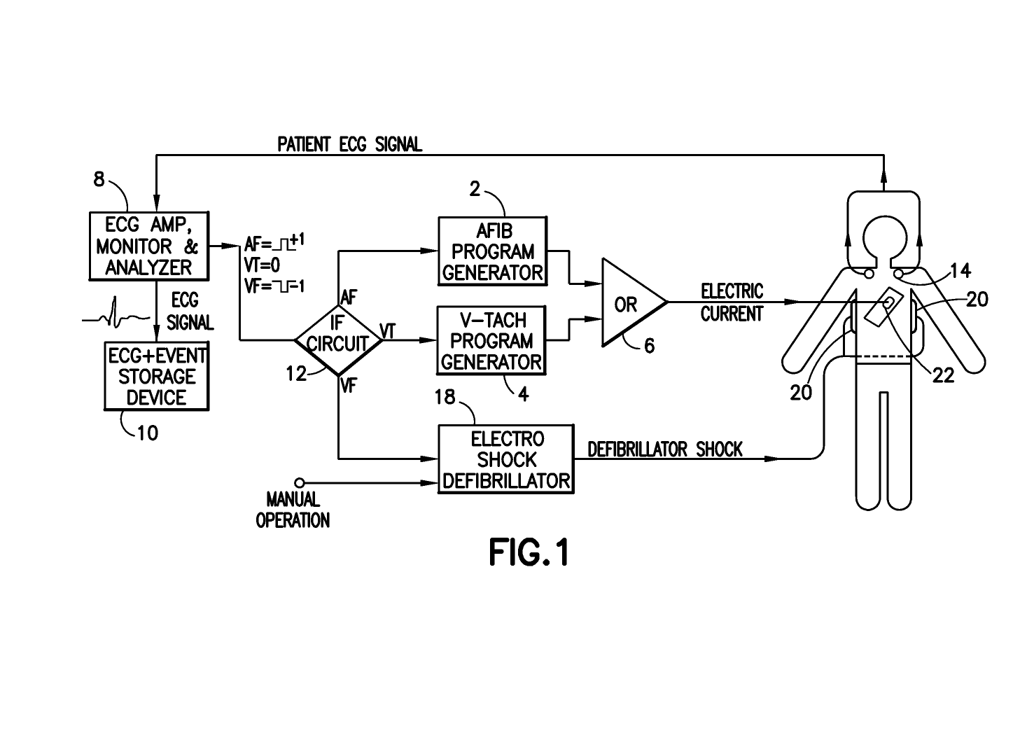

FIG. 1 shows the configuration of an apparatus for treating cardiac arrhythmia in accordance with one embodiment. This apparatus comprises an electromagnetic resynchronization (EMR) device (to be described in detail hereinafter) and a defibrillator 18. The EMR device, in turn, comprises an ECG monitoring system (items 8, 10 and 14 in FIG. 1) and an electromagnetic field generator (items 2, 4, 6, 12, 22).

The ECG monitoring system comprises a plurality of regular ECG electrodes 14, which are connected via electrode wires to an ECG amplifier, monitor and analyzer 8. This unit has in it the hardware and software to amplify and digitize the ECG signal picked up from the patient's body, and apply algorithms to detect and identify the different arrhythmias (e.g., atrial fibrillation, atrial flutter, ventricular tachycardia). The raw ECG data, along with data representing the results of ECG analysis, as well as event data representing the interpreted arrhythmias are stored continuously in a digital data storage device 10. This data is available for retrieval by a play back system to analyze a patient's ECG output. The ECG monitoring system can be programmed with the capability to determine the type of arrhythmia which is afflicting the patient.

The ECG amplifier, monitor and analyzer 8 will produce a logical output which is a function of the type of arrhythmia detected: +1 volt for atrial fibrillation or atrial flutter; 0 volt for ventricular tachycardia; and −1 volt for ventricular fibrillation. That logical output is input to a logic-level detection circuit 12 of the electromagnetic field generator. The logic-level detection circuit 12 detects the logic level of the incoming signal and outputs an activation signal to one of three devices in dependence on the detected logic level. If the logic level is +1 volt, the logic-level detection circuit 12 outputs an activation signal to an atrial fibrillation program generator 2. If the logic level is 0 volt, the logic-level detection circuit 12 outputs an activation signal to a ventricular tachycardia program generator 4. If the logic level is −1 volt, the logic-level detection circuit 12 outputs an activation signal to the defibrillator 18.

In response to an activation signal from the logic-level detection circuit 12, the atrial fibrillation program generator 2 sends pulses of electrical current to the coil or coil array 22 via OR gate 6, which pulses cause the coil or coil array 22 to produce a low-intensity magnetic field having a peak intensity in a first peak intensity range and a frequency in a first frequency range, which ranges are selected based on results of clinical treatment of atrial fibrillation. The electromagnetic field produced should have a frequency in a range of 5 to 22 Hz (inclusive) and a peak strength or intensity in a range of 10 picotesla to 10 nanotesla (inclusive) at the target location inside the patient's heart.

In response to an activation signal from the logic-level detection circuit 12, the ventricular tachycardia program generator 4 sends pulses of electrical current to the coil or coil array 22 via OR gate 6, which pulses cause the coil or coil array 22 to produce a low-intensity magnetic field having a peak intensity in a second peak intensity range and a frequency in a second frequency range which ranges are selected based on results of clinical treatment of ventricular tachycardia. The electromagnetic field produced by the ventricular tachycardia program generator 4 preferably has a frequency in a range of 10 to 60 Hz (inclusive) and a peak strength or intensity in a range of 900 picotesla to 25 nanotesla (inclusive) at the target location inside the patient's heart.

In response to an activation signal from the logic-level detection circuit 12, the defibrillator 18 will charge its capacitors to the appropriate pre-set voltage to deliver an electrical shock to the patient's heart via special electrodes incorporated in defibrillator pads 20 (shown attached to the patient in FIG. 1). When the defibrillator 18 is triggered, the system will produce an alarm signal (i.e., visual or audible) and a prerecorded voice message announcing that the patient is going to receive a defibrillator electrical shock. This system has also a manual operating button to be operated by skilled emergency teams to enable the delivery of an electrical shock if needed and the patient is unconscious. The success of a resuscitation from a sudden cardiac arrest depends on the time elapsed since the heart stopped pumping blood efficiently: more than 5 minutes means brain damage, more than 10 minutes means certain death. The regular defibrillator can save a patient's life if the resuscitation response time is short enough.

The system shown in FIG. 1 can be set-up and operated in the following manner (not necessarily in the order in which the steps are listed):

(1) The ECG electrodes 14 are attached to the patient's body.

(2) An EMR pad, incorporating a coil or coil array 22, is attached to the patient's chest as shown in FIG. 1.

(3) If needed, the defibrillator pads 20 are attached to the patient's body.

(4) An electrical cable from the ECG amplifier. monitor and analyzer 8 is connected to the ECG electrodes 14 so that the former can receive ECG signals from the patient.

(5) An electrical cable from the electromagnetic wave generator is connected to the coil or coil array 22 for delivery of pulsed electric current from one of the program generators 2 or 4 to the coil(s).

(6) If needed, an electrical cable from the defibrillator 18 is connected to the electrodes incorporated in pads 20.

(7) Operation of the ECG monitor and analyzer 8 is started. The analyzer has a display screen. The operator checks whether the ECG signal being displayed and recorded is clear. As soon as the operator has started the unit, the ECG data is stored in the ECG and event storage device 10.

(8) As explained in more detail below, the ECG monitor and analyzer 8 monitors the incoming ECG data and determines whether the ECG data indicates the occurrence of a cardiac event, such as atrial fibrillation, ventricular tachycardia or ventricular fibrillation. Upon determining that the patient is suffering from one of these conditions, the ECG monitor and analyzer 8 outputs a signal (+1 volt for atrial fibrillation, 0 volt for ventricular tachycardia, and −1 volt for ventricular fibrillation) to logic-level detection circuit 12, which in turn will send a triggering pulse to the appropriate program generator (the atrial fibrillation program generator 2 or the ventricular tachycardia program generator 4) or to the defibrillator 18.

(9) In response to an activation signal from the ECG monitor/analyzer 8 indicating that an atrial fibrillation event is occurring, the atrial fibrillation program generator 2 of the electromagnetic wave generator will generate a pulsed electric current that causes the coil or coil array 22 to generate a pulsed low-intensity magnetic field having a peak intensity in a first peak intensity range and a frequency in a first frequency range. In response to an activation signal from the ECG monitor/analyzer 8 indicating that a ventricular tachycardia event is occurring, the ventricular tachycardia program generator 2 of the electromagnetic wave generator will generate a pulsed electric current that causes the coil or coil array 22 to generate a pulsed low-intensity magnetic field having a peak intensity in a second peak intensity range and a frequency in a second frequency range. The first and second ranges, for each parameter, may overlap. In either case, the OR gate 6 will deliver the pulsed electric current to the coil or coil array 22 to initiate the beneficial effect of the applied electromagnetic field.

(10) The ECG monitor/analyzer 8 will detect once the arrhythmia has ceased and signal the electromagnetic field generator to stop. If the arrhythmia has not ceased, the ECG monitor/analyzer will signal the electromagnetic field generator to change frequency (±5 Hz) for another session. If during the second therapeutic session, the arrhythmia still does not cease, the ECG monitor/analyzer will signal the patient to see his doctor or change the therapeutic routine.

(11) in case that, following the initiation of the EMR activity, the patient's heart transitions into ventricular fibrillation, the defibrillator is instructed to deliver an electric shock.

The atrial fibrillation program generator 2 and ventricular tachycardia program generator 4 may comprise separate processors or a single processor that executes respective software modules. The ECG monitor and analyzer 8 may comprise a separate processor capable of executing commercially available programs designed to detect the occurrence of the cardiac conditions of interest. Alternatively, the program generators and the ECG monitor/analyzer may be embodied as one computer or processor that hosts the various ECG analysis and field generation programs.

The ECG and event storage device 10 (which may also comprise a separate processor) provides the ability to play back the ECG signals received and analyzed by the monitor/analyzer 8. The ECG and event storage device 10 will also record the time and date each time the EMR device or the defibrillator is triggered and what information was sent to the logic-level detection circuit 12.

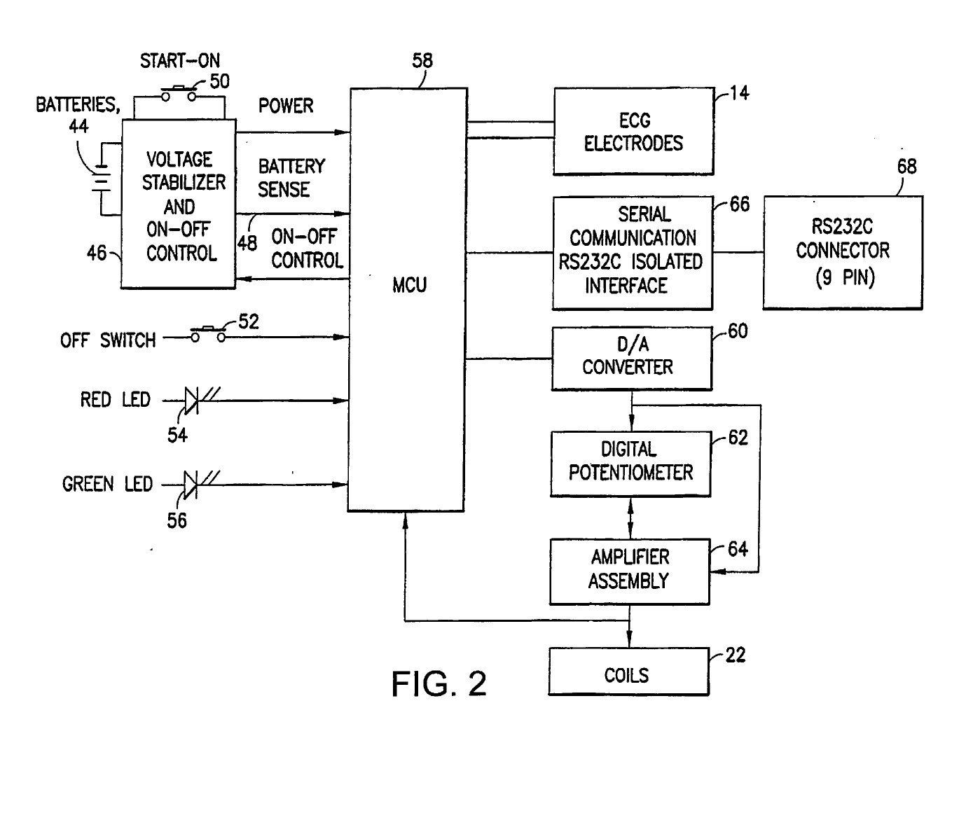

FIG. 2 shows the circuitry of a battery-powered integrated EMR unit in accordance with an alternative embodiment, which unit can be programmed to perform all of the functions of the ECG monitor/analyzer and the electromagnetic field generator shown in FIG. 1. This device can be used by humans as a non-invasive pacemaker to suppress arrhythmia. This EMR unit can be lightweight and wearable by a cardiac patient. In accordance with this alternative embodiment, the EMR unit can communicate with a separate defibrillator in the event that the EMR therapy induces ventricular fibrillation.

Referring to FIG. 2, the integrated EMR unit comprises a microcontroller unit (MCU) 58 having an ND input coupled to at least one ECG electrode 14 attached to the chest of a patient. The microcontroller 58 may be programmed with ECG analysis software for detecting predetermined points on the ECG waveforms acquired by the ECG electrode 14. The microcontroller 58 incorporates non-volatile memory (e.g., battery-powered memory, flash memory or other non-volatile memory technology) for storing also waveform/protocol parameters and other data received from a master or host computer. Such waveform/protocol parameters may include some or all of the following: gain, amplitude, frequency, waveshape, duration of treatment, time of treatment, number of times a treatment may be repeated, and other relevant functions, such as amplitude modulation, frequency modulation and phase modulation. These functions may be programmed to depend on the results of the ECG analysis. Alternatively, a microcomputer or microprocessor having similar functionality can be used.

The battery-powered unit shown in FIG. 2 further comprises an RS232C communications channel by means of which waveform parameters and treatment protocol data can be loaded into the microcontroller from a computer. The channel comprises serial communication RS232C isolated interface 66 and an RS232C 9-pin connector 68.

The microcontroller 58 processes the loaded treatment parameters and outputs a digital signal representing a waveform having a desired frequency and shape for driving the coils 22 of the magnetic field transducer. A digital-to-analog (D/A) converter 60 converts the digital signals output by the microcontroller 58 into an analog signal having the desired frequency and waveshape. The microcontroller 58 also outputs a digital value representing a setting to a digital potentiometer 62. The function of the digital potentiometer 62 is to adjust the level of the treatment signal, since the D/A converter 60 is always giving full amplitude. The output of the D/A converter 60 and the digital potentiometer 62 form the input signal to the amplifier assembly 64, the output of which is the current applied to the coils 22.

The microcontroller 58 outputs the digital waveform signals in accordance with the stored treatment protocol data. For example, the treatment protocol may comprise a single continuous treatment or a plurality of treatment cycles separated by quiescent intervals or rest periods.

Still referring to FIG. 2, the microcontroller 58 is powered by a battery or batteries 44. The voltage from the battery is supplied to the microcontroller 58 via a voltage stabilizer/on-off control circuit or chip 46. The voltage supplied by the battery is stabilized by the voltage stabilizer. The on-off control portion of chip 46 receives a control signal from the microcontroller 58. The treatment device can turn itself off by command from the microcontroller. The output of the analog chain (i.e., the D/A converter 60, the digital potentiometer 62 and the amplifier assembly 64) is connected into an ND input of the microcontroller 58 to enable autotest of the proper operation of that subsystem. A Start-On pushbutton 50 is provided to turn the system on (after it is shut down). An Off pushbutton 52 is also provided for shutting down the system at any time. More precisely, the microcontroller 58 is programmed to send an Off command to chip 46 in response to pushbutton 52 being depressed. Optionally, the microcontroller can be programmed to take some other action in response to depression of pushbutton 52, in which case the latter could serve as a function switch in certain situations.

Still referring to FIG. 2, numeral 48 indicates a low-voltage sense circuit that outputs an analog signal proportional to the current battery voltage to an input of the microcontroller 58. The microcontroller 58 incorporates an ND converter that converts the analog signal to a digital value. That digital value is compared to a stored threshold value. When the battery voltage falls to a level corresponding to the stored threshold value, the microcontroller causes the red LED 54 to blink, indicating that the battery needs to be replaced. The red LED 54 is turned on as long as the EMR device is activated. A green LED 56 is activated whenever the speaker is used and blinks when treatment is being performed. The green LED lights continuously for one minute after the end of treatment whenever number of available treatments remaining is either one or two.

The waveform parameters and treatment protocol data may be fed to the microcontroller 58 via the RS232C interface. Alternative communications channels can be employed. All parameters and protocol data are stored in a central computer and loaded into microcontroller 58 either directly or via a PC computer connected to the treatment device. The microcontroller 58 can store any desired waveform by receiving a series of values that can be repeatedly transmitted as an amplitude and time interval as selected by data transferred from the master computer. Alternatively, the microcontroller can have an internal algorithm to generate a waveform of the desired shape, amplitude and frequency to be supplied to the coils.

In accordance with one implementation, the ECG analysis software loaded into the microcontroller 58 analyzes the ECG data from the ECG electrode and when a cardiac arrhythmia event is detected, generates a command which enables software for generating the appropriate pulsed low-intensity magnetic field. More specifically, the microcontroller 58 can be programmed to generate: (a) a first pulsed low-intensity magnetic field having a peak intensity in a first peak intensity range and a frequency in a first frequency range in response to detection of an atrial fibrillation event; or (2) second pulsed low-intensity magnetic field having a peak intensity in a second peak intensity range and a frequency in a second frequency range in response to detection of a ventricular tachycardia event.

The most common cardiac arrhythmia, atrial fibrillation, occurs when the normal electrical impulses that are generated by the SA node are overwhelmed by disorganized electrical impulses in the atria. These disorganized impulses cause the muscles of the upper chambers of the heart to quiver (fibrillate) and this leads to the conduction of irregular impulses to the ventricles. On an ECG there are two major characteristics that identify atrial fibrillation: (1) No P-waves before the QRS on the ECG. This is because there are no coordinated atrial contractions. (2) The heart rate will be irregular. Irregular impulses that the ventricles are receiving cause the irregular heart rate. When the heart rate is extremely rapid, it may be difficult to determine if the rate is irregular, and the absence of P-waves will be the best indicator of atrial fibrillation.

Previous algorithms have relied upon tracking either the absence of a type of electrical activity in the heart known as the P-wave, or the variability in the timing of the contraction of the ventricle (which produces the tall spikes on an ECG tracing). While absence of P-wave fluctuations is the most telling barometer for atrial fibrillation, motion and noise artifacts can result in atrial fibrillation going undetected.

The system disclosed herein will use a commercially available diagnostic software module for detecting atrial fibrillation and atrial flutter. One known software program, written in MATLAB, can detect portions of a patient's electrocardiogram that have characteristics of atrial fibrillation or atrial flutter. This is achieved by using the RR-intervals of the ECG data. Atrial fibrillation detection can be based on statistical techniques, such as root mean squares of successive differences, turning points ratio and Shannon entropy. For atrial flutter detection, a time-frequency analysis of the patient data can be implemented.

Tachycardia/tachyarrhythmia is defined as a rhythm with a heart rate greater than 100 bpm. An unstable tachycardia exists when cardiac output is reduced to the point of causing serious signs and symptoms. Serious signs and symptoms commonly seen with unstable tachycardia are: chest pain, signs of shock, shortness of breath, altered mental status, weakness, fatigue, and syncope. The system disclosed herein will use a commercially available diagnostic software module for detecting tachycardia.

While apparatus for treating cardiac arrhythmias have been described with reference to various embodiments, it will be understood by those skilled in the art that various changes may be made and equivalents may be substituted for elements thereof without departing from the scope of the teachings herein. In addition, many modifications may be made to adapt the teachings herein to a particular situation without departing from the scope thereof.

As used in the claims, the term “computer system” should be construed broadly to encompass a system having at least one computer or processor, and which may have multiple computers or processors that communicate through a network or bus. As used in the preceding sentence, the terms “computer” and “processor” both refer to devices comprising a processing unit (e.g., a central processing unit) and some form of memory (i.e., computer-readable medium) for storing a program which is readable by the processing unit.

The method claims set forth hereinafter should not be construed to require that the steps recited therein be performed in alphabetical order (alphabetical ordering in the claims is used solely for the purpose of referencing previously recited steps) or in the order in which they are recited. Nor should they be construed to exclude any portions of two or more steps being performed concurrently or alternatingly.

1. A method, performed by a computer system, of therapeutically treating a patient, comprising the following steps:
storing one or more treatment protocols in the computer system for treating one or more types of arrhythmia;
analyzing in the computer system ECG signals received from ECG electrodes placed on a patient to determine whether the patient is experiencing an arrhythmia and, if so, which type of arrhythmia the patient is experiencing; and
if the arrhythmia is of a type for which a treatment protocol is stored in the computer system, driving one or more electrically conductive coils placed near the patient's heart with electrical current to cause said coils to generate a magnetic field,
wherein in the event that the arrhythmia was determined to be atrial fibrillation, said generated magnetic field has a frequency in a range of 5 to 22 Hertz and a peak intensity, in the volume occupied by the patient's heart, in a range of 10 picotesla to 10 nanotesla; and
wherein in the event that the arrhythmia was determined to be ventricular tachycardia, said generated magnetic field has a frequency in a range of 10 to 60 Hertz and a peak intensity, in the volume occupied by the patient's heart, in a range of 900 picotesla to 25 nanotesla.

2. The method as recited in claim 1, further comprising the step of synchronizing the driving of said coils with a predetermined point on an acquired ECG waveform or a predetermined point on an ECG waveform derived from one or more acquired ECG waveforms.

3. The method as recited in claim 1, wherein said driving of said coils is triggered and gated to deliver electric current during only a part of the patient's ECG cycle.

4. The method as recited in claim 3, wherein said part of patient's ECG cycle is the QRS period.

5. The method as recited in claim 1, wherein the generated magnetic field is focused in a region of the SA node of the patient's heart.

6. (canceled)

7. (canceled)

8. The method as recited in claim 1, wherein in the event that the arrhythmia was determined to be ventricular fibrillation, said method further comprises delivering an electrical shock to the patient's heart.

9. The method as recited in claim 1, further comprising:
varying the frequency at which said coils are driven;
analyzing the ECG signals to determine which frequency produced the optimum response in the patient; and
driving said coils to produce a magnetic field having said optimum frequency for a predetermined duration.

10. A system for therapeutic treatment of patients, comprising:
one or more electrically conductive coils placed near the patient's heart;
ECG electrodes attached to the patient; and
a computer system coupled to said coils and said ECG electrodes, said computer system being programmed to execute the following operations:
analyzing the ECG signals to determine whether the patient is experiencing an arrhythmia and, if so, which type of arrhythmia the patient is experiencing; and
if the arrhythmia is of a type for which a treatment protocol is stored in the computer system, driving said coils with electrical current to cause said coils to generate a magnetic field, wherein in the event that the arrhythmia was determined to be atrial fibrillation, the generated magnetic field has a frequency in a range of 5 to 22 Hertz and a peak intensity, in the volume occupied by the patient's heart, in a range of 10 picotesla to 10 nanotesla, and in the event that the arrhythmia was determined to be ventricular tachycardia, said generated magnetic field has a frequency in a range of 10 to 60 Hertz and a peak intensity, in the volume occupied by the patient's heart, in a range of 900 picotesla to 25 nanotesla

[symbol]

11. The system as recited in claim 10, wherein said computer system comprises:
a processor which produces a logical output which is a function of the type of arrhythmia detected; and
a logic-level detection circuit that detects the logic level of the logical output from said processor and then outputs an activation signal on a first output port if the detected logic level has a first value and outputs an activation signal on a second output port if the detected logic level has a second value.

12. The system as recited in claim 11, wherein said computer system further comprises an atrial fibrillation program generator coupled to said first output port and a ventricular tachycardia program generator coupled to said second output port.

13. The system as recited in claim 12, wherein said logic-level detection circuit outputs an activation signal on a third output port if the detected logic level has a third value, said system further comprising a defibrillator coupled to said third output port.

14. The system as recited in claim 10, wherein said computer system is further programmed to synchronize the driving of said coils with a predetermined point on an acquired ECG waveform or a predetermined point on an ECG waveform derived from one or more acquired ECG waveforms.

15. The system as recited in claim 10, wherein said computer system is further programmed to trigger and gate the driving of said coils to deliver electric current during only a part of the patient's ECG cycle.

16. The system as recited in claim 15, wherein said part of patient's ECG cycle is the QRS period.

17. The system as recited in claim 10, wherein the generated magnetic field is focused in a region of the SA node of the patient's heart.

18. (canceled)

19. (canceled)

20. The system as recited in claim 10, wherein said computer system is further programmed to execute the following operations:
varying the frequency at which said coils are driven;
analyzing the ECG signals to determine which frequency produced the optimum response in the patient; and
driving said coils to produce a magnetic field having said optimum frequency for a predetermined duration.

21. A system for therapeutic treatment of patients, comprising:
one or more electrically conductive coils for placement near the patient's heart;
ECG electrodes for attachment to the patient; and
a computer system coupled to said coils and said ECG electrodes, said computer system being programmed to execute the following operations:
analyzing the ECG signals to determine whether the patient is experiencing an arrhythmia and, if so, which type of arrhythmia the patient is experiencing; and
if the arrhythmia is of a type for which a treatment protocol is stored in the computer system, driving said coils with electrical current to cause said coils to generate a magnetic field having a frequency in a range of 2 to 60 Hertz and a peak intensity, in the volume occupied by the patient's heart, in a range of 10 picotesla to 25 nanotesla,
the computer system comprising:
a processor which produces a logical output which is a function of the type of arrhythmia detected;
a logic-level detection circuit that detects the logic level of the logical output from said processor and then outputs an activation signal on a first output port if the detected logic level has a first value and outputs an activation signal on a second output port if the detected logic level has a second value;
an atrial fibrillation program generator coupled to said first output port and a ventricular tachycardia program generator coupled to said second output port; and

[symbol]

22. The system as recited in claim 21, wherein said logic-level detection circuit outputs an activation signal on a third output port if the detected logic level has a third value, said system further comprising a defibrillator coupled to said third output port.

23. The system as recited in claim 22, wherein said computer system is further programmed to synchronize the driving of said coils with a predetermined point on an acquired ECG waveform or a predetermined point on an ECG waveform derived from one or more acquired ECG waveforms.

24. The system as recited in claim 23, wherein said computer system is further programmed to trigger and gate the driving of said coils to deliver electric current during only a part of the patient's ECG cycle.
